(19) United States
(12) Patent Application Publication
(10) Pub. No.: US 2005/0113286 A1
(43) Pub. Date: May 26, 2005

(54) Methods for treating congestive heart failure

(76) Inventors: George F. Schreiner, Los Altos, CA (US); Darlene Horton, San Francisco, CA (US); Santosh Vetticaden, San Ramon, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/931,498

(22) Filed: Aug. 31, 2004

Related U.S. Application Data
(63) Continuation-in-part of application No. 10/655,745, filed on Sep. 5, 2003.
(63) Continuation-in-part of application No. 10/390,546, filed on Mar. 18, 2003.
(60) Provisional application No. 60/364,736, filed on Mar. 18, 2002.

(51) Int. Cl. A61K 38/17

(52) U.S. Cl. 514/2

(57) ABSTRACT

The present invention relates to methods for administration of natriuretic peptide that are especially useful for treatment of a CHF patient. The methods are characterized by the serial and intermittent administration of a composition that provides a dose of natriuretic peptide. The methods of the invention are also useful for the treatment of chronic CHF patients, especially chronically decompensated CHF patients.

### CROSS REFERENCE

This application is a continuation-in-part of U.S. patent Ser. No. 10/390,546, filed Mar. 18, 2003, which claims the benefit of priority of priority of U.S. Provisional Patent Application No. 60/364,736, filed Mar. 18, 2002, both of which are hereby incorporated by reference.

### FIELD OF THE INVENTION

The invention is in the field of treating cardiovascular disorders. The methods provided herein are especially useful for treating chronic congestive heart failure.

### BACKGROUND OF THE INVENTION

Advanced congestive heart failure (HF) accounts for about 1 million hospital admissions yearly in the United States (US) and is associated with a 1-year mortality rate of 20%. American Heart Association. Heart Disease and Stroke Statistics-2004 Update, Dallas, Tex.: American Heart Association 2003, p 42. Patients at risk for hospitalization for HF likely have either new-onset or advanced disease. In patients with advanced disease, HF is classified as New York Heart Association [NYHA] Class II or IV. In the Class IV patients, the statistics are more grim, with the chance of rehospitalization within 6 months approaching 50% and 1-year mortality ranging between 40% and 70%. The American Heart Association estimates the 2004 direct costs of congestive heart failure in the US to be $26.7 Billion. American Heart Association. Heart Disease and Stroke Statistics-2004 Update. The cost of a single hospital admission primarily for HF in 2002 was $8,250, whereas the Diagnosis-Related Group (DRG) reimbursement from Medicare for that same year was only $4,989.⁽CMS National 2002 Inpatient Discharge Database (MEDPAR); data analyzed by HealthMarketInsights). This represents an economic burden for hospitals because Medicare is the primary payer for treatment of this disease condition.

Efforts to contain rising costs for hospital acute care have resulted in shorter hospital stays for patients with acutely decompensated HF. These shorter stays may not allow for adequate diuresis, titration of oral medications, or for patients to receive the full benefit of intravenously (IV) administered medications.

Furthermore, current adjunctive strategies that available for the management of advance HF are limited to ventricular replacement therapy (transplantation or left ventricular assist systems), protocol preparation, and referral to hospice. Accordingly alternative therapies that improve quality of life and/or reduce the number of hospital admissions for decompensated HF patients are needed. While there is increasing interest among clinicians in the use of outpatient IV therapy for the treatment of advanced HF, there is no consensus among clinicians regarding patient selection, dosing or treatment duration. The use of milrinone and dobutamine in this setting is controversial and may lead to increased mortality. (O'Connell, J. B., Clin. Cardiol. 23:III6-10 (2000)). Indeed, the ACC/AHA guidelines warn that long-term intermittent use of IV inotropes for treatment of left ventricular dysfunction is of “unproved value and not recommended.”⁽Hunt S A, Baker D W, Chin M H, Cinquegrani M P, Feldman A M, Francis G S, Ganiats T G, Goldstein S, Gregoratos G, Jessup M L, Noble R J, Packer M, Silver M A, Stevenson L W, Gibbons R J, Antman E M, Alpert J S, Faxon D P, Fuster V, Jacobs A K, Hiratzka L F, Russell R O, Smith S C Jr, American College of Cardiology/American Heart Association. ACC/AHA guidelines for the evaluation and management of chronic heart failure in the adult: executive summary: a report of the American College of Cardiology/American Heart Association Task Force on Practice Guidelines (Committee to revise the 1995 Guidelines for the Evaluation and Management of Heart Failure). J Am Coll Cardiol 2001;38:2101-2113. Yet, despite these guidelines, clinicians use inotropes in the outpatient setting because there is no currently approved alternative therapy. Thus, there is an unmet need for an adjunctive therapy for effectively managing advanced chronic HF.

### BRIEF SUMMARY OF THE INVENTION

The present invention relates the treatment of congestive heart failure, in particular, a method for the treatment of congestive heart failure in a subject in need thereof, wherein said method comprises the serial and intermittent administering of a therapeutically effective dose of natriuretic peptide to said subject. In a preferred embodiment, the invention involves the administering of a plurality of infusions of said natriuretic peptide to a subject with CHF over a period of not less than 2 to 4 weeks, each of said infusions being spaced apart over a period of 1 to 14 days. The methods of the invention are especially useful for treatment of a CHF patient who exhibits symptoms of decompensated congestive heart failure. The methods of the invention are useful for the treatment of chronic CHF patients, especially those patients exhibiting subacute but decompensated symptoms. Such a subject may be in need of near and long term management of CHF.

In a further embodiment, the invention is directed to a method for preventing the onset of acutely decompensated CHF, said method comprising the serial and intermittent administering of a therapeutically effective dose of natriuretic peptide to a subject with CHF wherein said administering comprises a plurality of infusions of said natriuretic peptide to a subject with CHF over a prolonged and therapeutically effective period.

In a related embodiment, the invention is directed to a method for preventing death or the need for hospitalization in a subject having CHF, said method comprising the serial and intermittent administering of a therapeutically effective dose of natriuretic peptide to a subject with CHF wherein said administering comprises a plurality of infusions of said natriuretic peptide to a subject with CHF over a prolonged and therapeutically effective period.

In another embodiment, the invention is directed to the long term treatment of CHF in a subject in need thereof, said treatment comprising the serial and intermittent administering of a therapeutically effective dose of natriuretic peptide to a subject with CHF wherein said administering comprises administering a plurality of infusions of said natriuretic peptide to said subject over a prolonged and therapeutically effective period. Preferably, said long term treatment results in the lowering of aldosterone and/or endothelin 1 concentrations in subjects having CHF wherein cardiac remodeling is prevented, reversed and/or ameliorated.

The invention and its associated embodiments are particularly useful in subjects with CHF who are at higher risk for hospitalization and/or death. These higher risk groups are defined as: subjects with an RAS (Risk Assessment Score)≧4; subjects defined as NYHA class III and/or class IV; and subjects identified as having renal insufficiency (elevated serum creatinine levels greater than about 1.5 mg/dL, preferably greater than about 2.0 mg/dL). Preferably said defined high risk groups comprise subjects with CHF who are defined as NYHA class IV or NYHA class III with elevated serum creatinine levels greater than about 2.0 mg/dL.

In a further embodiment, the methods of the invention are provided as the primary IV vasoactive therapy for a patient with CHF, and such patient is infused with therapeutically effective doses of natriuretic peptide for 4-6 hours weekly.

### BRIEF DESCRIPTION OF THE FIGURES

FIG. 1A is a chart identifying the frequency of selected adverse events (AEs) for all patients involved the FUSION trial through week 12. Renal AEs included increased blood urea nitrogen, increased serum creatinine, abnormal kidney function, acute kidney failure, and oliguria.

FIG. 1B is a chart identifying the frequency of selected adverse events (AEs) for CHF patients having an RAS score of ≧ through week 12. Renal AEs included increased blood urea nitrogen, increased serum creatinine, abnormal kidney function, acute kidney failure, and oliguria.

FIG. 2A is a chart that provides the change from baseline for aldosterone concentrations in the FUSION CHF patients after infusion of nesiritide. *p<0.02 compared to baseline

FIG. 2B is a chart that provides the change from baseline for endothelin-1 concentrations in the FUSION CHF patients after infusion of nesiritide. †p=0.008 compared to baseline.

### DETAILED DESCRIPTION OF THE INVENTION

Definitions

The term “ameliorate” denotes a lessening of an effect. To ameliorate a condition or disease refers to a lessening of the symptoms of the condition or disease.

An “individual” or “subject” is a vertebrate, preferably a mammal, more preferably a human.

“Mammal” refers to any animal classified as a mammal, including humans, domestic and farm animals, and zoo, sport, or pet animals, such as, for example, horses, sheep, cows, pigs, dogs, cats, etc. Preferably, the mammal is human.

A “therapeutically effective amount” or a “effective amount” is an amount sufficient to effect beneficial or desired results. Preferably, the effective amount is provided in multiple doses for a therapeutically effective period of time, such multiple doses and said desired period of time each being cumulatively sufficient to effect the beneficial or desired result.

A “therapeutically effective period” is a duration of administration that is sufficient to effect beneficial or desired results.

“Administration” means any manner of providing natriuretic peptide to a subject or patient. Routes of Administration can be accomplished through any means known by those skilled in the art. Such means include but are not limited injection, intravenous, oral, subcutaneous, intradermal and the like. Administration may also be accomplished through formulations and/or devices resulting in controlled and/or metered release of natriuretic peptide to a subject or patient. Such formulations and/or devices include those known to those skilled in the art. Administration “in combination with” one or more further therapeutic agents means any manner with provides for the beneficial effects of the administration of both agents, including simultaneous (concurrent) administration and consecutive administration in any order.

The term “modulate” means to control in a predictable fashion, either by increasing or by decreasing the targeted parameter, as indicated from the context.

A “treatment” is an approach for obtaining a beneficial or desired result, especially a clinical result, especially the administration of an agent to a subject for purposes which may include prophylaxis, amelioration, prevention or cure of an undesired physiological condition or disease. Such treatment need not necessarily completely ameliorate the condition or disorder. For purposes of this invention, beneficial or desired clinical results include, but are not limited to, alleviation of symptoms, diminishment of extent of tissue injury or disease, stabilized (i.e., not worsening) state of tissue injury or disease, delay or slowing of the progression or tissue injury or disease, amelioration or palliation of an undesired physiological condition or disease state, and remission (whether partial or total), whether detectable or undetectable. “Treatment” can also mean prolonging survival as compared to expected survival if not receiving treatment. “Treatment” is an intervention performed with the intention of preventing the development or altering the pathology of a disorder. Accordingly, “treatment” refers to both therapeutic treatment and prophylactic or preventative measures. Those in need of treatment include those already with the disorder as well as those in which the disorder is to be prevented.

“Prolonged administration” means Administration of natriuretic peptide to a subject or patient having CHF over a therapeutically effective period. Prolonged administration preferably involves a plurality of serial and intermittent intravenous infusions of natriuretic peptide to a subject or patient wherein the subject or patient receives at least one infusion of natriuretic peptide once every two weeks for a duration of at least two hours for each infusion.

“Progression of congestive heart failure” means death or the need for hospitalization of a patient afflicted with congestive heart failure. Without being limited to a particular theory or mechanism, the progression of the disease may ultimately result from compensatory remodeling of cardiac tissue, tissue scarring, neurohormonal imbalances and/or renal dysfunction. In any case and for purposes of this patent application, the endpoints for progression of congestive heart failure are hospitalization and/or death.

“Hospitalization” means the admission of a patient to a hospital setting during which the patient is treated for acutely decompensated heart failure. Hospitalization also includes Hospitalization equivalent.

“Hospitalization equivalent” means an unscheduled outpatient treatment for acutely decompensated heart failure wherein said treatment comprises the intravenous administration of one or more vasoactive drugs (e.g., dopabutamine, dopamine, milrinone, nitroglycerin, natriuretic peptide, and equivalents).

“Carriers” as used herein include pharmaceutically acceptable carriers, excipients, or stabilizers which are nontoxic to the cell or mammal being exposed thereto at the dosages and concentrations employed.

The term “pharmaceutically acceptable salt” as used herein refers to salt forms of a substance that are substantially non-toxic to living organisms. Typical pharmaceutically acceptable salts include those salts prepared by reaction of a desired agent, such as a desired form of natriuretic peptide, with a pharmaceutically acceptable mineral or organic acid or an inorganic base. Such salts are known as acid addition and base addition salts.

Acids commonly employed to form acid addition salts are inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of pharmaceutically acceptable salts formed from such acids are the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma.-hydroxybutyrate, glycollate, tartrate, methanesulfonate, propanesulfonate, naphthalene-1-sulfonate, naphthalene-2-sulfonate, mandelate, mesylate, and the like. Preferred pharmaceutically acceptable acid addition salts are those formed with mineral acids such as hydrochloric acid and hydrobromic acid, and those formed with organic acids such as maleic acid and methanesulfonic acid.

Salts of amine groups may also comprise quaternary ammonium salts in which the amino nitrogen carries a suitable organic group such as an alkyl, alkenyl, alkynyl, or aralkyl moiety.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, sodium carbonate, sodium bicarbonate, potassium bicarbonate, calcium hydroxide, calcium carbonate, and the like. The potassium and sodium salt forms are particularly preferred.

It should be recognized that the particular counterion forming a part of any salt of this invention is not of a critical nature, so long as the salt as a whole is pharmacologically acceptable and as long as the counterion does not contribute undesired qualities to the salt as a whole.

The term “essentially free of contaminants” refers to a substance that is purified to a degree such that the substance contains no, or acceptable levels of, undesired or unnecessary substances that arose form, or had been present during, the in vitro or in vivo synthesis of the desired substance.

NYHA classification refers to the following definitions of CHF patients:

New York Heart Association Functional Classification

| | |
|---|---|
| 1. | Patients with cardiac disease but without resulting limitations of |
| | physical activity. Ordinary physical activity does not cause undue |
| | fatigue, palpitation, dyspnea, or anginal pain. |
| 2. | Patients with cardiac disease resulting in slight limitation of |
| | physical activity. They are comfortable at rest. Ordinary physical |
| | activity results in fatigue, palpitation, dyspnea, or anginal pain. |
| 3. | Patients with cardiac disease resulting in marked limitation of |
| | physical activity. They are comfortable at rest. Less than ordinary |
| | physical activity causes fatigue, palpitation, dyspnea, or anginal |
| | pain. |
| 4. | Patients with cardiac disease resulting in inability to carry on any |
| | physical activity without discomfort. Symptoms of cardiac |
| | insufficiency or of the anginal syndrome may be present even at |
| | rest. If any physical activity is undertaken, discomfort is increased. |

Congestive Heart Failure (CHF or HF)

Congestive heart failure (CHF; cardiac failure) is a condition in which weakened heart function exists together with a build-up of body fluid. Cardiac failure often occurs when cardiac output is insufficient to meet metabolic demands of the body, or when the heart cannot meet the demands of operating at increased levels of filling/diastolic pressure. Therapy involves not only support of the weakened heart function but also treatment to counteract the build up of the body fluid.

Congestive heart failure may be caused by many forms of heart disease. Common causes of congestive heart failure include: narrowing of the arteries supplying blood to the heart muscle (coronary heart disease); prior heart attack (myocardial infarction) resulting in scar tissue large enough to interfere with normal function of the heart; high blood pressure; heart valve disease due to past rheumatic fever or an abnormality present at birth; primary disease of the heart muscle itself (cardiomyopathy); defects in the heart present at birth (congenital heart disease) and infection of the heart valves and/or muscle itself (endocarditis and/or myocarditis). Each of these disease processes can lead to congestive heart failure by reducing the strength of the heart muscle contraction, by limiting the ability of the heart's pumping chambers to fill with blood due to mechanical problems or impaired diastolic relaxation, or by filling the heart's chambers with too much blood.

Advanced congestive heart failure (CHF) includes both acute and chronic presentations. Typically, in both acute and chronic presentations, advanced CHF may involve patients who are decompensated. Patients presenting with acutely decompensated CHF usually have an acute injury to the heart, such as a myocardial infarction, mitral regurgitation or ventricular septal rupture. Typically, the injury compromises myocardial performance (for example, a myocardial infarction) or valvular/chamber integrity (for example, mitral regurgitation or ventricular septal rupture). Such injuries result in an acute rise in the left ventricular (LV) filing pressures. The rise in the LV filing pressures results in pulmonary edema and dyspnea. The treatment of patients with acutely decompensated CHF focuses on temporarily stabilizing the patient and/or morbidities associated with the patient's overall critical condition. This typically is accomplished by treating the immediate and apparent conditions associated with heart failure. In addition, the heart's function is supported by treatments to reduce LV filling pressures and to improve cardiac performance.

Patients with chronic decompensated CHF often have symptoms of volume overload and/or low cardiac output. These symptoms are associated with chronic LV systolic dysfunction.

Compositions Useful for the Invention

The invention provides methods of administration of pharmaceutically active compositions that are useful for both the prophylactic and therapeutic treatment of CHF patients, preferably CHF patients that are decompensated. The pharmaceutically active compositions of the invention are characterized as comprising a natriuretic peptide, sufficient to provide a therapeutically effective amount of a natriurertic peptide to such patient when administered in a therapeutically effective dose over a therapeutically effective period.

The natriuretic peptide that is present in a composition of the invention can be any of the family of therapeutically effective natriuretic peptides, or a mixture of the same. Examples of useful natriuretic peptides include, for example, atrial natriuretic peptide (ANP), brain natriuretic peptide (BNP or B-type natriuretic peptide) and C-type natriuretic peptide (CNP). Of them, ANP and BNP are preferred, and BNP is the most preferred. Sequences of many useful forms of natriuretic peptide are provided in (U.S. Patent Application Publication No. 20010027181A1, incorporated by reference herein).

Specific examples of ANPs that can be used in the methods of the invention include: human ANP (human atrial natriuretic peptide; hANP, Kangawa et al., Biochem. Biophys. Res. Commun., Vol. 118, p. 131, 1984) (Seq. ID No. 1) or rat ANP (Kangawa et al., Biochem. Biophys. Res. Commun., Vol. 121, p. 585, 1984). Such ANPs comprise 28 amino acids. Such ANPs may be administered as a peptide having a ring structure of ANP (formation of a disulfide bond based on Cys), and a C-terminal portion succeeding the ring structure. An example of such a peptide is a peptide having amino acid residues at the 7-position to the 28-position of ANP is provided in U.S. Patent Application Publication No. 20010027181A1. Another example is frog ANP. Of them, human ANP (hANP), and especially recombinant hANP is particularly preferred.

Specific examples of BNPs that can be used in the methods of the invention include human BNP (hBNP). Human BNP comprises 32 amino acids and involves the formation of a disulfide bond, like the above-described ANP (Sudoh et al., Biochem. Biophys. Res. Commun., Vol. 159, p. 1420, 1989). See also, U.S. Pat. Nos. 5,114,923, 5,674,710, 5,674,710, 5,948,761, each of which is hereby incorporated by reference. Various BNP's of the origin other than human, such as pig BNP and rat BNP, are also known, and can be used similarly. A further example is chicken BNP.

Specific examples of CNPs that can be used in the methods of the invention include pig CNP. Pig CNP comprises 22 amino acids and involves the formation of a disulfide bond, like the above-described ANP and BNP (Sudoh et al., Biochem. Biophys. Res. Commun., Vol. 168, p. 863, 1990) (human and rat also have the same amino acid sequence), chicken CNP (Arimura et al., Biochem. Biophys. Res. Commun., Vol. 174, p. 142, 1991). Frog CNP (Yoshihara et al., Biochem. Biophys. Res. Commun., Vol. 173, p. 591, 1990) can also be used.

Furthermore, any person skilled in the art can apply modification, such as deletion, substitution, addition or insertion, and/or chemical modification to amino acid residues in the amino acid sequence of a known natriuretic peptide (e.g., the aforementioned human ANP; hANP), as desired, by a known method. One skilled in the art can confirm that the resulting compound is a compound which has the activity of acting on a receptor of the starting ANP or BNP or CNP. Derivatives having this activity, therefore, are included in the substance as an active ingredient which is administered to a patient in accordance with the method of the present invention.

A substance that activates the patient's natriuretic peptide receptor could also be used in the compositions of the invention in place of, or in addition to, one or more of the natriuretic peptides discussed above. Such substance should be capable of acting on a natriuretic peptide receptor to increase intracellular cGMP production. Such substances may be non-peptide compounds.

With respect to compositions that are useful for the invention, the natriuretic peptide is preferably provided as a free (non-salt) form, or as a pharmaceutically acceptable salt. A salt with an inorganic acid preferably includes salts with hydrochloric acid, sulfuric acid, and phosphoric acid. The salt with an organic acid thus may, preferably be, for example, acid addition salts with formic acid, acetic acid, butyric acid, succinic acid, and citric acid. The salt is preferably in the form of a metal salt with sodium, potassium, lithium or calcium, or a salt with an organic base.

To produce compositions for infusion, carriers or additives can be added to provide a desired stability or property to the composition. Examples of such carriers and additives include: (1) tonicity agents such as sodium chloride, D-mannitol, and D-sorbitol, (2) pH regulators such as hydrochloric acid and citric acid, (3) buffering agents such as sodium citrate, sodium acetate, and boric acid, and (4) soothing agents such as procaine hydrochloride; as well as stabilizers, and surface active agents. Often the physiologically acceptable carrier is an aqueous pH buffered solution. Examples of physiologically acceptable carriers include buffers composed of phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS®.

In consideration of the stability, etc. of the active natriuretic peptide ingredient, it can be selected whether the active ingredient should be formed into a preparation to be used after dissolution or suspension when required, or into a liquid preparation.

The Methods of the Invention

According to the invention, serial and intermittent administration of natriuretic peptides provides a safe, therapeutic and prophylactic benefit to CHF patients. In a preferred embodiment, CHF patients who are in a chronically decompensated state are administered serial and intermittent infusions to achieve the desired treatment. In comparison to the term “acutely decompensated”, by “chronically decompensated” it is meant that the CHF patient, at the time of administration of a therapeutic and/or prophylactic dosage of a natriuretic peptide according to the present invention, is not exhibiting critical symptoms of acute heart failure which require immediate treatment and pose the threat of imminent death if not immediately treated in an acute (i.e. hospital) setting. Such patients may include, for example, patients in New York Heart Association functional classifications I through IV as described herein, as well as patients who may have recently been hospitalized for previous treatment of acute heart failure. Preferably, the peptide dosage can range to where the serial and intermittent infusion(s) is less than about 0.01 μg/kg/min.

The pharmaceutically active compositions that provide the active natriuretic peptide are preferably administered to the patient who is in need of the same in the form of an injection. Such injections can be, for example, intravenous, intramuscular, subcutaneous, intradermal, intrasternal, intraperitoneal or intra-articular. Most preferably, the compositions are provided in the form of an infusion, and especially, an intravenous infusion. The peptide may also be administered through alternative delivery routes (i.e. oral, transdermal, subcutaneous, and the like) and controlled release formulations that are available to those skilled in the art and are adapted so as to achieve the desired therapeutic effect as provided herein.

In a preferred embodiment, therapeutically effective doses of natriuretic peptides and especially, nesiritide (commercially sold as Natrecor®) are used in the compositions of the invention in a form intended for injection or infusion. Preferably, such compositions reduce pulmonary capillary wedge pressure and ameliorate dyspnea in patients who are at rest or with minimal activity.

The infusion can be administered for any effective period of time, for example, 1, 2, 3, 4, 5, 6, 7, 8, 9 or 10 hours, or a desired period of time in between. In a further embodiment, infusions for greater than 10 hours are performed. In a preferred embodiment, a patient in need of such treatment is infused for 4-6 hours. Preferably such infusion is continuous although two infusions of shorter duration following one right after the other may be used.

The infusion rate may be any that is tolerated by the patient. Higher or lower infusion rates can be used if desired. In a preferred embodiment, the infusion rate is about 0.00125 μg/kg/min to about 0.01 μg/kg/min. In a further preferred embodiment, an infusion rate of about 0.005 μg/kg/min is used.

The infusion rate should be sufficient to provide a therapeutically effective amount of the natriuretic peptide during the infusion period or treatment protocol but without compromising patient safety.

In one embodiment, a separate initial bolus of a preparation that contains natriuretic peptide is administered to the patient followed by a more sustained infusion. Such a bolus preferably provides from about 0.25, 0.5, 0.75, 1.0, 1.25, 1.5 or 1.75 μg/kg natriuretic peptide.

The optimal volume of the infusion and amount of the active natriuretic peptide will vary by body weight. For example, a 30 kg (66 pound) patient might first be given a 2.5 ml bolus of 0.5 μg/ml of the natriuretic peptide composition infused at 0.8 ml/hr so as to provide for 0.0025 μg/kg/min, followed by a minimum bolus of 0.25 μg/kg in 1.3 mL infused at 0.4 mL/hr and 0.00125 μg/kg/min to a maximum bolus of 1.0 μg/kg in 5.0 mL infused at a maximal rate of 1.5 mL/hr to provide 0.005 μg/kg/min.

For another example, a 30 kg (66 pound) patient might first be given a 5.0 ml bolus of 1.0 μg/ml of the natriuretic peptide composition infused at 1.5 ml/hr so as to provide for 0.005 μg/kg/min, followed by a minimum bolus of 0.5 μg/kg in 2.5 mL infused at 0.8 mL/hr and 0.0024 μg/kg/min.

In contrast, a 175 kg (386 pound) patient might first be given a 14.6 ml bolus of 0.5 μg/ml of the natriuretic peptide composition infused at 4.4 ml/hr so as to provide for 0.0025 μg/kg/min, followed by a minimum bolus of 0.25 μg/kg in 7.3 mL infused at 2.2 mL/hr and 0.00124 μg/kg/min to a maximum bolus of 1.0 μg/kg in 29.2 mL infused at a maximal rate of 8.8 mL/hr to provide 0.005 μg/kg/min.

Alternatively, a 175 kg (386 pound) patient might first be given a 29.2 ml bolus of 1.0 μg/ml of the natriuretic peptide composition infused at 8.8 ml/hr so as to provide for 0.005 μg/kg/min, followed by a minimum bolus of 0.5 μg/kg in 14.6 mL infused at 4.4 mL/hr and 0.0025 μg/kg/min.

In a preferred embodiment, the methods of the invention provide therapeutically effective doses of natriuretic peptides, and especially, nesiritide, to a CHF patient in need of long term management and/or at high risk of hospitalization and/or death. CHF patients considered to be at such high risk include subjects with an RAS (Risk Assessment Score)≧4; subjects defined as NYHA class III and/or class IV; and subjects identified as having renal insufficiency (elevated serum creatinine levels greater than about 1.5 mg/dL, preferably greater than about 2.0 mg/dL). Preferably said defined high risk groups comprise subjects with CHF who are defined as NYHA class IV or NYHA class III with elevated serum creatinine levels greater than about 2.0 mg/dL. For purposes of this invention, RAS (Risk Assessment Score) is a means for characterizing CHF patients with respect to known prognostic factors for hospitalization or death. RAS is quantitated by the absence or presence of each of the following 7 risk factors: (1) serum creatinine of >2.0 mg/dl in the preceding 30 days; (2) NYHA class IV for the preceding 60 days; (3)≧65 years old; (4) history of sustained ventricular tachycardia; (5) ischemic etiology of HF; (6) diabetes; and (7) outpatient use of nesiritide or inotropic agents in preceding 6 months. CHF patients with an RAS≧4 present with 4 or more of the 7 risk factors identified above).

Treatment preferably occurs in a subacute or outpatient setting. Infusion of the composition of the invention is therapeutically effective if it provides a desirable hemodynamic and neurohormonal effects in addition to maintaining the advantageous safety profile of the administered natriuretic peptide.

In another embodiment, the method provided by the invention is useful as an add-on therapy to oral medications. In a preferred embodiment of the method of the invention, serial and intermittent IV infusions of a natriuretic peptide, and especially of nesiritide are provided at therapeutically effective doses to patients who are also being treated with oral therapeutic agents to manage such patient's CHF, such therapeutically effective doses of the infused natriuretic peptide resulting at least in part in a more rapid and sustained compensation of CHF in patients with frequent episodes of acutely decompensated CHF, and/or who are in need of chronic CHF treatment in a subacute or outpatient setting.

It is an advantage of the invention that the method of the invention can be used to treat and/or manage CHF patients who are in a chronically decompensated state.

Having now generally described the invention, the same will become better understood by reference to certain specific examples which are included herein for purposes of illustration only and are not intended to be limiting unless other wise specified. All referenced publications and patents are incorporated, in their entirety by reference herein.

### Experimental

The following experimental section provides the first study undertaken for repeated infusions of nesiritide administered in an outpatient setting for the post-hospitalization management of chronic decompensated HF, and is the largest controlled study of outpatient IV infusion therapy conducted in patients with CHF. The experimental section should not be considered to limit the embodiments of the invention as contemplated herein.

Nesiritide, a recombinant form of human B-type natriuretic peptide (BNP), has been approved for the treatment of patients with acutely decompensated HF. Its pluripotent properties, which include desirable hemodynamic, neurohormonal, lusitropic, renal, and reverse remodeling effects, are advantageous in HF. (Burger A J, Horton DP, LeJemtel T, Ghali J K, Torre G, Dennish G, Koren M, Dinerman J, Silver M, Cheng M L, Elkayam U. Effect of nesiritide (B-type natriuretic peptide) and dobutamine on ventricular arrhythmias in the treatment of patients with acutely decompensated congestive heart failure: the PRECEDENT study. Am Heart J 2002;144:1102-1108. Clarkson P B, Wheeldon N M, Macleod C, Coutie W, MacDonald T M. Brain natriuretic peptide: effect on left ventricular filling patterns in healthy subjects. Clin Sci (Lond) 1995;88:159-164. Colucci W S, Elkayam U, Horton D P, Abraham W T, Bourge R C, Johnson A D, Wagoner L E, Givertz M M, Liang C, Neibaur M, Haught W H, LeJemtel T H, for the Nesiritide Study Group. Intravenous nesiritide, a natriuretic peptide, in the treatment of decompensated congestive heart failure. N Engl J Med 2000;343:246-253. Publication Committee for the VMAC Investigators. Intravenous nesiritide vs nitroglycerin for treatment of decompensated congestive heart failure: a randomized controlled trial. JAMA 2002;287:1531 1540. Burger A J, Elkayam U, Neibaur M T, Haught H, Ghali J, Horton D P, Aronson D. Comparison of the occurrence of ventricular arrhythmias in patients with acutely decompensated congestive heart failure receiving dobutamine versus nesiritide therapy. Am J Cardiol 2001;88:35-39. Tamura N, Ogawa Y, Chusho H, Nakamura K, Nakao K, Suda M, Kasahara M, Hashimoto R, Katsuura G, Mukoyama M, Itoh H, Saito Y, Tanaka I, Otani H, Katsuki M, Nakao K. Cardiac fibrosis in mice lacking brain natriuretic peptide. Proc Natl Acad Sci USA 2000;97:4239-4244. Sakata Y, Yamamoto K, Masuyama T, Mano T, Nishikawa N, Kuzuya T, Miwa T, Hori M. Ventricular production of natriuretic peptides and ventricular structural remodeling in hypertensive heart failure. J Hypertens 2001; 19: 1905-1912.) Nesiritide has been well tolerated in controlled clinical trials involving more than 1,200 patients with HF, and prospective randomized comparisons to active therapies have shown that its properties compare favorably with those of IV nitroglycerin.(Burger A J, Horton D P, LeJemtel T, Ghali J K, Torre G, Dennish G, Koren M, Dinerman J, Silver M, Cheng ML, Elkayam U. Effect of nesiritide (B-type natriuretic peptide) and dobutamine on ventricular arrhythmias in the treatment of patients with acutely decompensated congestive heart failure: the PRECEDENT study. Am Heart J 2002;144:1102-1108. Publication Committee for the VMAC Investigators. Intravenous nesiritide vs nitroglycerin for treatment of decompensated congestive heart failure: a randomized controlled trial. JAMA 2002;287:1531-1540.) In the Vasodilation in the Management of Acute CHF (VMAC) trial, nesiritide resulted in a more rapid resolution of the symptoms of acute decompensated HF than did either IV diuretics or nitroglycerin. In separate investigations, nesiritide did not engender ventricular rhythm disturbances as did dobutamine and it was associated with a shorter length of stay, fewer readmissions, and a commensurate pharmacoeconomic benefit when compared with milrinone. (Lewis D A, Gurram N R, Abraham W T, Akers W S. Effect of nesiritide versus milrinone in the treatment of acute decompensated heart failure. Am J Health Syst Pharm 2003;60(suppl 4):S16-S20.) Nesiritide thus represents a potential adjunct to established usual medical therapy for HF and theoretically may provide a more reasonable alternative to intermittent inotropic infusions in the management of patients with chronic decompensated HF who are at high risk for rehospitalization.

Methods

Study Objective

The objective of the FUSION I study was to assess the safety and tolerability of nesiritide when administered in an outpatient setting as serial parenteral infusions to patients with chronic decompensated HF who were at high risk for hospitalization and were receiving established usual therapy for HF. The study compared usual care with usual care plus nesiritide given as 1 of 2 dosing strategies. All patients were evaluated weekly and treated in established HF clinics.

Study Design

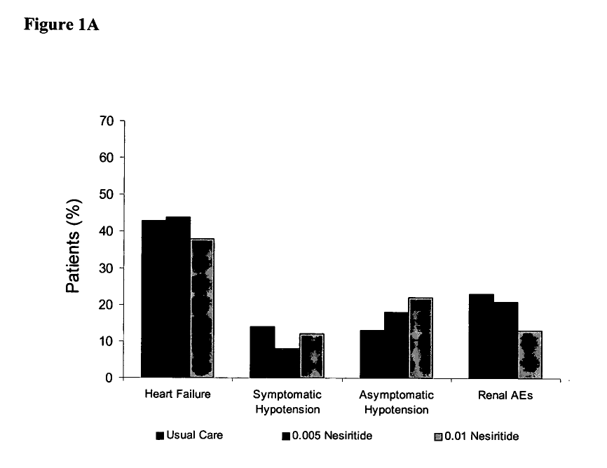

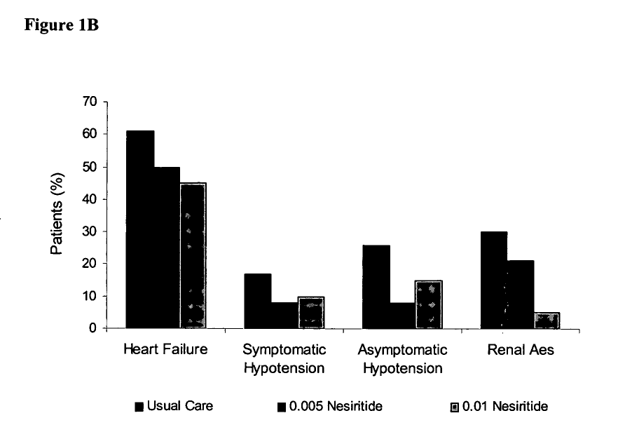

In this multicenter, open-label, pilot study, patients were randomly assigned to 1 of 3 treatment groups in a 1:1:1 ratio: (1) usual care, as determined by the investigating physician; (2) usual care plus nesiritide 0.005 μg/kg/min given for 4 to 6 hours, preceded by a 1.0-μg/kg bolus; or (3) usual care plus nesiritide 0.01 μg/kg/min given for 4 to 6 hours, preceded by a 2.0-μg/kg bolus (FIG. 1). The study treatment period was 12 weeks with an additional 4 weeks of follow-up. Nesiritide (Natrecor®) was supplied by Scios Inc., Fremont, Calif. Informed consent was obtained from each patient prior to enrollment, and the Ethics Committee of each institution prospectively approved the study.

Selection of Study Participants

Eligible patients were at least 18 years of age, had HF classified as NYHA class III or IV for ≧60 days prior to randomization, had a 6-minute walk test result of <400 m, and had at least 2 hospital admissions (or unscheduled outpatient visits requiring IV vasoactive treatment) for acutely decompensated HF within the preceding 12 months. At least 1 of the hospital admissions or unscheduled visits had to have occurred within the preceding 5 to 30 days. All participating patients were receiving optimal treatment for HF with long-term oral medications, as tolerated.

Patients were not eligible for the study if they had a systolic blood pressure below 90 mm Hg; had undergone placement of a biventricular pacemaker in the preceding 60 days; had undergone placement of an implantable cardioverter-defibrillator (ICD) in the preceding 30 days; were receiving chronic dialysis or were likely to require dialysis during the 4-month study period; had evidence of acute myocardial infarction within the preceding 30 days; were unable to complete a 6-minute walk test; or had received or were awaiting an organ transplantation.

Study Medications

All patients, regardless of treatment assignment, were required to have weekly clinic visits in an established HF clinic. If the patient had been randomized to nesiritide, half of the protocol-specified nesiritide dose was administered on the first weekly visit to assess safety. The 0.005 μg/kg/min nesiritide group received a 0.5 μg/kg bolus followed by a 0.0025 μg/kg/min infusion; and the 0.01 μg/kg/min nesiritide group received a 1.0 μg/kg bolus followed by a 0.005 μg/kg/min infusion. At subsequent visits (starting with week 2), the dose was increased to the protocol-specified dose for that group.

Nesiritide was administered as an IV bolus immediately followed by a fixed-rate infusion for 4 to 6 hours, based on the investigators' discretion, for 12 consecutive weeks. Before each infusion, the investigator assessed the patient's hydration status and HF symptoms as per the protocol. The investigator could choose the infusion frequency from once every other week to twice weekly based on hydration status and HF symptoms.

Patients in all treatment groups were allowed to receive any long-term cardiac or noncardiac medications, including IV diuretics, according to investigator discretion. At each visit, the investigator ensured that optimal medical treatment regimens were established. Patients who were randomized to usual care only were allowed to receive positive inotropic agents (milrinone, dobutamine, or dopamine) only if the investigator believed that inotropic support was required to alleviate symptomatic HF and yield hemodynamic stability. Inotropic agents were not permitted in the nesiritide groups unless they were urgently required to prevent hospitalization. During hospitalization, there was no restriction on the use of inotropic agents or nesiritide, regardless of treatment group.

Study End Points and Measurements

The primary goal of this open-label study was to assess the safety and tolerability of different nesiritide doses administered as serial outpatient infusions. Safety and tolerability were measured through investigator reporting of adverse events (AEs), serious AEs, discontinuations of study infusions, laboratory assessments, and vital signs. An AE was defined as any new onset or worsening of any medical condition since study entry. A serious AE were those AEs, which lead to death, hospitalization, or prolonged hospitalization. The effect of nesiritide infusions on renal function was also monitored as a safety variable, with renal AEs defined as increased blood urea nitrogen (BUN) or serum creatinine levels (determined by investigator's discretion), onset of oliguria, or occurrence of acute renal failure. Clinical events were followed to further evaluate safety and tolerability.

Surrogate measures with mechanistic implications included measures of serum aldosterone, serum endothelin-1, and serial measurements of ventricular function using ejection fractions derived from echocardiography. Pre-infusion aldosterone and endothelin-1 concentrations were collected and analyzed at a core lab (The Mayo Clinic, Rochester, Minn.) at weeks 1, 4, 8, and 12; post-infusion concentrations were collected at weeks 1 and 12. Left ventricular ejection fractions were serially measured and read at a core lab (Midwest Heart Foundation Echocardiographic Core Laboratory, Lombard, Ill.).

At weeks 4, 8, and 12, the change from baseline global clinical status was assessed by both the patient and the investigator; assessments were rated on a 7-point ordinal scale from markedly worse (−3) to markedly better (+3).^{1l,12} In addition, patients' assessment of quality of life was measured at baseline and at weeks 4, 8, and 12 using the Minnesota Living With HF questionnaire. (Rector TS, Kubo SH, Cohn J N. Patients' self-assessment of their congestive heart failure. Part 2: Content, reliability and validity of a new measure, The Minnesota Living with Heart Failure Questionnaire. Heart Failure 1987;October/November:198-209.)

Statistical Considerations

The empirically determined, pre-planned sample size of 210 patients was to provide useful safety and tolerability information on the infusions and to establish event-rate estimates for future clinical trials.

To characterize treatment groups with respect to known prognostic factors for hospitalization or death, patients were prospectively stratified using a Risk Assessment Score (RAS). Two levels of risk were defined: patients with an RAS≧4 (i.e., with 4 or more of the 7 risk factors) and patients with an RAS<4. Risk factors utilized were: (1) serum creatinine of >2.0 mg/dl in the preceding 30 days; (2) NYHA class IV for the preceding 60 days; (3)≧65 years old; (4) history of sustained ventricular tachycardia; (5) ischemic etiology of HF; (6) diabetes; and (7) outpatient use of nesiritide or inotropic agents in preceding 6 months.

All randomized and treated patients were included in the intent-to-treat analyses. Comparisons of treatment groups with respect to baseline variables were assessed with the omnibus F test, the Kruskal-Wallis test, or the generalized Fisher's exact test, as appropriate.

The effect of treatment was assessed with a 2-way ANOVA, an F test, a stratified Wilcoxon test, a Wilcoxon test, or the generalized Fisher's exact test, depending on the distribution of the variable. Kaplan-Meier estimates of the survival curves for morbidity/mortality time-to-event data were calculated. A log-rank or stratified log-rank test was used for comparisons between 2 treatment groups.

Results

Patient Characteristics

Between December 2001 and February 2003, 210 patients from 46 United States study centers were enrolled and treated. Patients were randomly assigned to receive usual care only (n=69), usual care plus nesiritide 0.005 μg/kg/min (n=72), or usual care plus nesiritide 0.01 μg/kg/min (n=69).

The only significant difference in baseline medical history between the treatment groups was an increased prevalence seen in the usual care group of atrial fibrillation or atrial fibrillation/flutter at baseline (p=0.02). Otherwise, there were no significant differences in demographic and baseline characteristics among the 3 treatment groups (Table 1). The mean age was 66.9 years, and the majority of patients were male and white. More than 20% of patients had a history of moderate or severe renal disease, and 50% had diabetes. Approximately 1/3 of patients had a single- or dual-chamber pacemaker, and 25% had an ICD in place. Sixty-five percent and 35% had disease classified as NYHA class III and IV, respectively. At randomization, 32% of all patients had an RAS≧4. The distribution of RAS cohorts was similar among the 3 treatment groups. Twenty-three (33%), 24 (33%), and 20 (29%) of the usual care, 0.005 μg/kg/min nesiritide, and 0.01 μg/kg/min nesiritide groups, respectively, were in the RAS≧4 cohort.

Concomitant cardiovascular medications for HF treatment at baseline for the overall population included oral diuretics (99%), P-adrenergic blocking agents (74%), angiotensin-converting enzyme (ACE) inhibitors (61%), spironolactone (42%), and angiotensin II receptor blockers (ARBs) (19%) (Table 2). There was no significant difference in chronic oral therapy observed between the groups.

Nesiritide Dosing

Nesiritide patients received a total of 1,645 infusions for a mean (±SD) cumulative duration of 55±25 hours over the 12-week study period. During the study period, 39 patients each in the 0.005- and 0.01-μg/kg/min nesiritide groups (54% and 57%, respectively) skipped an infusion at least once owing to clinical evidence of euvolemia and compensated HF. Because of persistent evidence of volume overload and decompensated HF, 24 (33%) and 20 (29%) patients in the 2 dose groups, respectively, received at least 2 infusions during at least 1 of the 12 weeks. Although infusions could be skipped for clinical reasons or during hospitalizations, 111 nesiritide patients (79%) received an infusion near the end of the study period, in either week 11 or 12.

Use of Inotropic Agents

During the 2 weeks prior to randomization, 41% of patients had received an IV inotropic agent in an outpatient setting (Table 2). During the study, 40 patients (58%) in the usual-care-only group received an IV inotropic agent during at least 1 study visit because of clinical evidence of decompensated HF. By comparison, only 2 patients who received nesiritide (<2%) also received an inotropic agent during a study visit because of clinical evidence of decompensated HF.

General Safety

Of the 1,645 nesiritide infusions that were administered, 1,628 (99%) were completed per protocol; 11 (<1%) were discontinued because of an AE (Table 3). Of these 11 infusions, 10 were discontinued after the patient experienced an AE (2, symptomatic hypotension; 4, asymptomatic hypotension; 1 each, angina pectoris, myocardial infarct, nausea, and dehydration) and 1 was stopped after the patient experienced 2 AEs (nausea and dizziness). Six infusions (<1%) were discontinued for administrative reasons (Table 3). Thirteen patients (4 usual care, 7 nesiritide 0.005 μg/kg/min, and 2 nesiritide 0.01 μg/kg/min) were discontinued from the study before week 12 because of an AE, primarily worsening HF.

Overall, the frequency of AEs through 12 weeks was similar across treatment groups. The most frequently reported AEs for all patients were decompensated HF (42%), asymptomatic hypotension (18%), dyspnea (17%), and symptomatic hypotension (11%). There was no increase in the frequency of observed AEs in either nesiritide group compared with the usual-care group. FIG. 1 provides a summary of selected cardiovascular and renal AEs in all patients, as well as for the RAS≧4 cohort. The use of nesiritide in patients with RAS≧4 was not associated with an increase in observed adverse cardiovascular or renal events.

Clinical Outcomes

For the usual-care group compared with each nesiritide dose group and the all nesiritide group, there were no statistically significant differences in deaths or hospitalizations (Table 4). Forty (58%) usual-care patients and 67 (48%) nesiritide patients either were hospitalized or died (or both) during the 12-week treatment period (p=0.185). Seven (10%) usual-care patients and 9 (6%) nesiritide patients died (p=0.314), and 37 (54%) usual-care and 65 (46%) nesiritide patients were hospitalized (p=0.378). The predominant causes of death were HF and cardiac arrest; in no case was death considered related to study medication. Nesiritide patients showed trends for increasing days alive and out of the hospital compared with usual-care patients (p=0.131) (Table 4).

Within the RAS≧4 cohort, nesiritide resulted in a statistically significant increase in days alive and out of the hospital compared to the usual care group (p=0.027) (Table 4). Eighteen (78%) usual care and 23 (52%) nesiritide patients in the RAS≧4 cohort either died or were hospitalized through week 12 (p=0.038). Of these, 4 (17%) and 2 (5%) usual-care and nesiritide patients, respectively, died (p=0.079), and 17 (74%) and 22 (50%) usual-care and nesiritide patients, respectively, were hospitalized (p=0.072).

Clinical outcomes also were evaluated in the subgroups of patients with each of the 7 RAS factors. Elevated serum creatinine, >2.0 mg/dL, was associated with a statistically significant increase in days alive and out of hospital in response to therapy with nesiritide compared to usual care alone. NYHA class IV status was associated with a trend towards a reduction in hospitalizations with nesiritide compared with usual care (Table 5).

Clinical Status

All treatment groups reported improvement (marked or moderate) in global clinical status during this short-term study. According to investigators' assessments, the nesiritide 0.01-μg/kg/min group showed a significant improvement compared with the usual-care group (p≦0.004 at all time points). By the patients' assessments of clinical status, there were no significant differences among treatment groups (Table 6).

All treatment groups reported a significant improvement in quality of life at weeks 4, 8, and 12 compared with baseline as assessed by the Minnesota Living With HF questionnaire. There were no significant differences between treatment groups.

Neurohormones

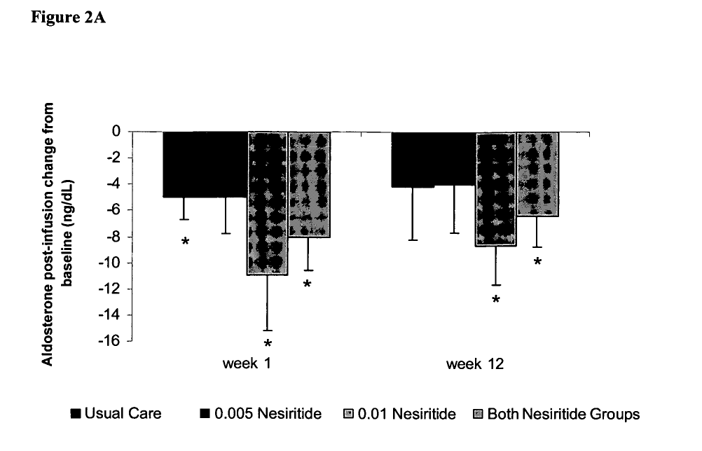

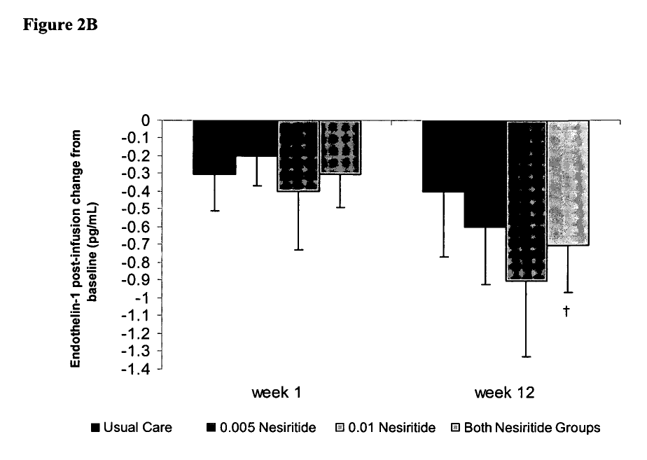

Mean pre-infusion aldosterone values showed no significant changes over time in any treatment group. Mean post-infusion values of aldosterone at weeks 1 and 12 showed significant declines from baseline in both nesiritide groups. Mean changes from baseline (±standard deviation) at week 1 were: −5.0±5.9 pg/mL, −5.0±14.8 pg/mL, and −10.9±22.8 pg/mL in the usual care (p=0.01 compared to baseline), 0.005-μg/kg/min nesiritide, and 0.01-μg/kg/min nesiritide (p=0.02 compared to baseline) groups, respectively. Post-infusion levels of aldosterone declined overall, with significant changes from baseline after the week 12 infusion for the 0.01-μg/kg/min nesiritide the all-nesiritide group (FIG. 2A).

Mean changes (±standard deviation) in post-infusion concentrations of endothelin-1 from baseline to week 8 were −0.3±1.1 pg/mL, −0.2±1.5 pg/mL, and −0.6±2.0 pg/mL in the usual care, 0.005-μg/kg/min nesiritide, and 0.01-μg/kg/min nesiritide (p=0.04 compared to baseline) groups, respectively. Post-infusion levels of endothelin-1 declined overall, with a significant change from baseline after the week 12 infusion for the all-nesiritide group (FIG. 2B).

Ejection Fraction

The mean changes in ejection fraction from baseline to week 12 were +3.2±3.8%, +4.0±3.3%, and +5.3±5.0% in the usual-care, nesiritide 0.005-μg/kg/min, and nesiritide 0.01-μg/kg/min groups, respectively (p=0.03 for the nesiritide 0.01-μg/kg/min group compared with the usual-care group).

Discussion

This study was a randomized open-label pilot study that evaluated the safety of usual care with weekly visits versus usual care plus nesiritide administered serially over 12 weeks in an outpatient setting in patients with chronic decompensated HF. The study evaluated the utility of outpatient nesiritide infusions as a post-hospitalization strategy for an HF population already receiving optimal medical therapy and at high risk for rehospitalization. The study population was maximally treated at study entry, with a high percentage of patients receiving β adrenergic blocking agents, renin-angiotensin blockers (ACE inhibitors or ARBs), or spironolactone. The study population was representative of a population of significantly ill HF patients who are not likely candidates for transplantation or left ventricular device strategies due to age, psychosocial issues, and significant comorbidities.

In addition, this study population represents a group not usually evaluated in randomized controlled trials in HF because of their clinical instability. The acuity of this patient population was evident in the 3-month combined-event rate of death or hospitalization of 51% in all patients and 61% in the RAS≧4 cohort. By comparison, the event rates in this trial, when extrapolated to a 1-year period, approach the severity of illness seen in the optimal medical management arm, which included 70% use of inotropes, of the Randomized Evaluation of Mechanical Assistance for the Treatment of Congestive Heart Failure (REMATCH) trial. (Rose E A, Gelijns A C, Moskowitz A J, Heitjan D F, Stevenson L W, Dembitsky W, Long J W, Ascheim D D, Tierney A R, Levitan R G, Watson J T, Meier P, Ronan N S, Shapiro P A, Lazar R M, Miller L W, Gupta L, Frazier O H, Desvigne-Nickens P, Oz M C, Poirier V L. Long-term mechanical left ventricular assistance for end-stage heart failure. N Engl J Med 2001;345:1435-1443.)

The intent of this pilot trial was to determine the safety and tolerability of outpatient nesiritide infusions as a treatment for chronic decompensated HF. This trial demonstrated that nesiritide infusions at both doses tested were similarly well tolerated in the outpatient setting; less than 1% of the 1,645 infusions administered were discontinued for AEs, with no AE being consistently responsible for termination of the infusions. Cardiovascular AEs, including symptomatic hypotension, and renal AEs were not increased in the nesiritide groups compared to the usual-care group. The RAS≧4 cohort treated with nesiritide tended to have a lower incidence of cardiovascular or renal AEs compared with those treated with usual care.

There were no statistically significant differences in observed clinical outcomes in the 3 groups studied. In the RAS≧4 patients, however, those treated with nesiritide had statistically significantly fewer deaths or hospitalizations compared with those treated with usual care only. There is at least one physiologic mechanism that supports a clinical benefit with nesiritide. Nesiritide is known to result in a decrease in left ventricular filling pressures, and follow-up of hospitalized patients with advanced HF has shown that a reduction in pulmonary capillary wedge pressure is one of the most important predictors of improved outcomes. (Fonarow G C, Hamilton Mass., Moriguchi J, Creaser J W, Rourke D A. Hemodynamic predictors of clinical outcome in decompensated advanced heart failure [abstract 038]. J Card Fail 2001;3 (suppl 2):13.)

It is reasonable to note that the unstable cohort of patients (i.e., the patients prospectively identified as RAS≧4 by well-accepted criteria) safely tolerated the serial outpatient administration of nesiritide as adjunctive therapy for HF. The observation that the greatest relative reduction in events occurred in patients who met the risk criteria represented by NYHA class IV status and renal insufficiency suggests that the invention is particularly useful within this patient profile.

Data from this study suggest that patients who received nesiritide infusions experienced additional neurohormonal antagonism via reductions in aldosterone and endothelin-1. Recent studies have clearly implicated aldosterone as an important contributor to ventricular remodeling, arrhythmias, and mortality associated with left ventricular dysfunction. (Pitt B, Zannad F, Remme W J, Cody R, Castaigne A, Perez A, Palensky J, Wittes J, for the Randomized Aldactone Evaluation Study Investigators. The effect of spironolactone on morbidity and mortality in patients with severe heart failure. N Engl J Med 1999;341:709-717. Pitt B, Remme W, Zannad F, Neaton J, Martinez F, Roniker B, Bittman R, Hurley S, Kleiman J, Gatlin M, for the Eplerenone Post-Acute Myocardial Infarction Heart Failure Efficacy and Survival Study Investigators. Eplerenone, a selective aldosterone blocker, in patients with left ventricular dysfunction after myocardial infarction. N Engl J Med 2003;348:1309-1321. Cozza EN, Foecking M F, Vila M C, Gomez-Sanchez C E. Adrenal receptors for natriuretic peptides and inhibition of aldosterone secretion in calf zona glomerulosa cells in culture. Acta Endocrinol (Copenh) 1993;129:59-64.) The mild improvements in ejection fraction observed in the nesiritide group are the first data to support the potential role of natriuretic peptides in promoting reverse remodeling in chronic heart failure.

Although the data may suggest that the 0.005-μg/kg/min dose of nesiritide conferred benefit over the 0.01-μg/kg/min dose, this conclusion is confounded by several factors. First, several patients in each dose group either missed infusions or received 2 infusions in a given week, blurring the differences in total exposure to nesiritide. Second, 4 (6%), 7 (10%), and 2 (3%) patients in the usual-care, nesiritide 0.005-μg/kg/min, and nesiritide 0.01-μg/kg/min groups, respectively, discontinued the study prematurely due to an AE (primarily worsening HF), although included in the intent-to-treat analysis. Third, 3 usual-care patients and 3 nesiritide patients were lost to follow-up by week 12. Five of these patients withdrew their informed consent at the time of their premature withdrawal from the study; thus, follow-up events that occurred after consent was withdrawn could not be collected.

Study Limitations

The study was limited by its open-label design, the relatively small number of patients in each treatment group, and the relatively short treatment duration. This trial was not intended or powered to identify meaningful clinical end points. Its sole intent was to determine the safety, tolerability, and feasibility of outpatient nesiritide administration in this setting. Although the definition of “usual care” was left to the discretion of the investigator, it is possible that the safety profile observed for nesiritide patients reflect the adverse influence of inotropic agents in more than half of the usual-care patients. It is also notable that almost all nesiritide patients who were receiving outpatient inotropes at baseline were able to successfully discontinue the inotrope and tolerate serial infusions of nesiritide. Both the presence of an open-label design and the potential for investigator bias toward administration of inotropes confounds the clinical event data.

The results of this study should be interpreted in the context of structured care in a disease management program. It is likely that the improvements seen in quality-of-life indicators and the 6-minute walk test in all treatment groups are testimony to the positive benefits of weekly care and evaluation by HF disease management teams in established HF clinics. These outpatient disease management programs, albeit effective, are resource intensive and reflect tertiary care models that may be difficult to duplicate on a broad scale.

Conclusions

The results of this study demonstrate that serial outpatient infusions of nesiritide administered over 12 weeks to patients with chronic decompensated HF who were at high risk for rehospitalization were safe and well tolerated. Serial outpatient infusions of nesiritide led to acute reductions in aldosterone and endothelin and may have promoted reverse remodeling. Patients identified as NYHA class IV status and with significant renal dysfunction showed the greatest potential for benefit from nesiritide treatment, with apparent signals consistent with a decrease in hospitalizations and/or death, no increase in adverse cardiovascular events, and a trend towards fewer adverse renal events. Data from this study suggest that outpatient administration of nesiritide to patients with chronic HF may be a safe strategy and may represent a potentially beneficial clinical adjunct to standard medical therapy.

Having now fully described the invention, the same will be understood by those with skill in the art that the scope may be performed with a wide and equivalent range of conditions, parameters, and the like, without affecting the spirit or scope of the invention or any embodiment thereof.

TABLE 1
Demographic and Baseline Characteristics

| | Usual | Nesiritide (μg/kg/min) | | |
|---|---|---|---|---|
| | Care | 0.005 | 0.01 | All Patients |
| Characteristic | (n = 69) | (n = 72) | (n = 69) | (n = 210) |
| Age (mean ± SD) | 66.5 ± 11.80 | 66.5 ± 12.02 | 67.8 ± 14.28 | 66.9 ± 12.69 |
| Range | 39-87 | 44-89 | 22-87 | 22-89 |
| Gender - male | 51 (74%) | 49 (68%) | 46 (67%) | 146 (70%) |
| Race - white | 52 (75%) | 62 (86%) | 54 (78%) | 168 (80%) |
| Primary etiology of CHF | | | | |
| Ischemic | 38 (55%) | 53 (74%) | 40 (58%) | 131 (62%) |
| Idiopathic,dilated | 20 (29%) | 13 (18%) | 18 (26%) | 51 (24%) |
| cardiomyopathy | | | | |
| Other | 11 (16%) | 6 (8%) | 11 (16%) | 28 (13%) |
| Cardiovascular history | | | | |
| Hypertension | 53 (77%) | 56 (78%) | 54 (78%) | 163 (78%) |
| CAD | 55 (80%) | 57 (79%) | 54 (78%) | 166 (79%) |
| Previous MI | 40 (58%) | 46 (64%) | 40 (58%) | 126 (60%) |
| Atrial fib or fib/flutter* | 39 (57%) | 25 (35%) | 36 (52%) | 100 (48%) |
| Moderate/severe renal disease | 16 (23%) | 17 (24%) | 14 (20%) | 47 (22%) |
| Baseline creatinine (mg/dl) | 1.8 ± 0.70 | 1.8 ± 0.71 | 1.8 ± 0.69 | 1.8 ± 0.70 |
| (mean ± SD) | | | | |
| Diabetes | 31 (45%) | 41 (57%) | 34 (49%) | 106 (50%) |
| Insulin dependent | 16 (23%) | 22 (31%) | 19 (28%) | 57 (27%) |
| Non-insulin dependent | 15 (22%) | 19 (26%) | 15 (22%) | 49 (23%) |

CAD = coronary artery disease;
CHF = congestive heart failure;
fib = fibrillation;
MI = myocardial infarction;
SD = standard deviation.
*p = 0.02 between groups.

TABLE 2
Concomitant Cardiovascular Medication Use

| | Usual | Nesiritide (μg/kg/min) | | |
|---|---|---|---|---|
| Cardiovascular | Care | 0.005 | 0.01 | All Patients |
| Medication | (n = 69) | (n = 72) | (n = 69) | (n = 210) |

Baseline cardiovascular regimen*

| | | | | |
|---|---|---|---|---|
| Diuretics | 69 (100%) | 72 (100%) | 68 (99%) | 209 (99%) |
| β Blockers | 46 (67%) | 52 (72%) | 57 (83%) | 155 (74%) |
| ACE inhibitors | 39 (57%) | 49 (68%) | 41 (59%) | 129 (61%) |
| Non-IV nitrates | 33 (48%) | 33 (46%) | 35 (51%) | 101 (48%) |
| Spironolactone | 26 (38%) | 27 (38%) | 35 (51%) | 88 (42%) |
| ARB | 19 (28%) | 10 (14%) | 11 (16%) | 40 (19%) |
| IV inotropic agent* | | | | |
| Milrinone | 16 (23%) | 17 (24%) | 18 (26%) | 51 (24%) |
| Dobutamine | 8 (12%) | 7 (10%) | 4 (6%) | 19 (9%) |
| Dopamine | 5 (7%) | 5 (7%) | 6 (9%) | 16 (8%) |

During study visits through week 12

| | | | | |
|---|---|---|---|---|
| IV inotropic agentst† | 40 (58%) | 0 | 2 (3%) | 42 (20%) |
| IV diuretics | 48 (70%) | 42 (58%) | 44 (64%) | 134 (64%) |
| Nesiritide | 3 (4%) | 72 (100%) | 69 (100%) | 144 (69%) |

During hospitalizations through week 12‡

| | | | | |
|---|---|---|---|---|
| IV inotropic agents† | 17 (25%) | 15 (21%) | 14 (20%) | 46 (22%) |
| Nesiritide | 10 (14%) | 18 (25%) | 12 (17%) | 40 (19%) |

ACE = angiotensin-converting enzyme;
ARB = angiotensin II receptor blocker; IV = intravenous.
*Administered as an outpatient infusion during the 2 weeks prior to randomization.
†Inotropic agents included milrinone, dobutamine, or dopamine.
‡Administered at least once.

TABLE 3
Nesiritide Infusion Tolerability - All Nesiritide Treated Patients

| | | | All |
|---|---|---|---|
| | Nesiritide (μg/kg/min) | | Nesiritide |
| | 0.005 | 0.01 | Patients |
| | (n = 72) | (n = 69) | (n = 141) |
| Total number of infusions | 819 | 826 | 1645 |
| Infusion completed | 814 (99%) | 814 (99%) | 1628 (99%) |
| Infusions stopped due to | 4 (<1%) | 7 (<1%) | 11 (<1%) |
| adverse event | | | |
| Infusions stopped for | 1 (<1%) | 5 (<1%) | 6 (<1%) |
| administrative reasons | | | |
| Patients with infusion stopped | 4 (6%) | 5 (7%) | 9 (6%) |
| due to adverse event | | | |

TABLE 4
Clinical Outcomes Through 12 Weeks

| | | Nesiritide (μg/kg/min) | | |
|---|---|---|---|---|
| | | | | Both |
| | | | | Nesiritide |
| Clinical Outcome | Usual Care | 0.005 | 0.01 | Groups |
| All patients | n = 69 | n = 72 | n = 69 | n = 141 |
| All-cause death and | 40 (58%) | 33 (46%) | 34 (49%) | 67 (48%) |
| hospitalization | | | | |
| p value*^{†} | NA | 0.175 | 0.385 | 0.185 |
| Deaths | 7 (10%) | 6 (8%) | 3 (4%) | 9 (6%) |
| p value*^{†} | NA | 0.692 | 0.194 | 0.314 |
| All-cause hospitalization | 37 (54%) | 32 (44%) | 33 (48%) | 65 (46%) |
| p value*^{†} | NA | 0.314 | 0.610 | 0.378 |
| Days alive and out of hospital | | | | |
| Mean ± SD | 73.9 ± 17.8 | 76.1 ± 15.3 | 78.9 ± 10.9 | 77.5 ± 13.3 |
| 25^{th} percentile | 73.8 | 74.2 | 79.0 | 77.6 |
| p value* | NA | 0.253 | 0.159 | 0.131 |
| RAS ≧4 patients | n = 23 | n = 24 | n = 20 | n = 44 |
| All-cause death and | 18 (78%) | 10 (42%) | 13 (65%) | 23 (52%) |
| hospitalization | | | | |
| p value*^{†} | NA | 0.017 | 0.323 | 0.038 |
| Deaths | 4 (17%) | 1 (4%) | 1 (5%) | 2 (5%) |
| p value*^{†} | NA | 0.146 | 0.213 | 0.079 |
| All-cause hospitalization | 17 (74%) | 10 (42%) | 12 (60%) | 22 (50%) |
| p value*^{†} | NA | 0.039 | 0.515 | 0.072 |
| Days alive and out of hospital | | | | |
| Mean ± SD | 67.2 ± 22.3 | 76.3 ± 16.8 | 77.2 ± 14.0 | 76.7 ± 15.5 |
| 25^{th} percentile | 61.2 | 74.6 | 75.3 | 75.0 |
| p value* | NA | 0.028 | 0.128 | 0.027 |

NA = not applicable;
SD = standard deviation;
RAS = risk assessment score.
*Compared with usual care.
^{†}Time-to-event analysis by Kaplan-Meier estimates.

TABLE 5
Clinical Outcomes by Baseline Serum Creatinine Level > 2.0 mg/dL or NYHA Class IV

| | | Nesiritide (μg/kg/min) | | |
|---|---|---|---|---|
| | | | | Both |
| | | | | Nesiritide |
| | Usual Care | 0.005 | 0.01 | Groups |
| Lowest serum creatinine >2.0 mg/dL | | | | |
| during 30 days | | | | |
| prior to the study | n = 14 | n = 13 | n = 14 | n = 27 |
| Patient deaths | 4 (29%) | 1 (8%) | 1 (7%) | 2 (7%) |
| Patients hospitalized | 11 (79%) | 6 (46%) | 8 (57%) | 14 (52%)* |
| Days alive and out of hospital | | | | |
| Mean ± SD | 65.6 ± 20.7 | 75.7 ± 19.0 | 78.4 ± 9.0 | 77.1 ± 14.7† |
| Median | 76 | 84 | 81 | 81 |
| Baseline NYHA class IV | n = 26 | n = 25 | n = 22 | n = 47 |
| Patient deaths | 4 (15%) | 2 (8%) | 1 (5%) | 3 (6%) |
| Patients hospitalized | 18 (69%) | 12 (48%) | 12 (55%) | 24 (51%)‡ |
| Days alive and out of hospital | | | | |
| Mean ± SD | 68.5 ± 24.5 | 73.3 ± 18.4 | 77.7 ± 13.5 | 75.4 ± 16.2§ |
| Median | 80 | 84 | 82 | 83 |

NYHA = New York Heart Association.
*p = 0.176 compared with usual care.
†p = 0.019 compared with usual care.
‡p = 0.148 compared with usual care.
§p = 0.182 compared with usual care.

TABLE 6
Investigators' and Patients' Assessments of Global Clinical Status

| | | | Nesiritide (μg/kg/min) | | |
|---|---|---|---|---|---|
| | | | | | Both |
| | | | | | Nesiritide |
| | | Usual Care | 0.005 | 0.01 | Groups |
| Assessment | Week | (n = 69) | (n = 72) | (n = 69) | (n = 141) |

Investigators' assessment

| | | | | | |
|---|---|---|---|---|---|
| Reported improvement* | 4 | 58 (22%) | 63 (29%) | 62 (47%) | 125 (38%) |
| from baseline (%) | 8 | 49 (33%) | 50 (54%) | 54 (59%) | 104 (57%) |
| | 12 | 49 (37%) | 51 (55%) | 51 (59%) | 102 (57%) |
| Mean change from baseline | 4 | 0.5 ± 1.25 | 0.9 ± 1.28 | 1.1 ± 1.28† | 1.0 ± 1.28† |
| | 8 | 0.7 ± 1.24 | 1.3 ± 1.35† | 1.6 ± 1.19† | 1.4 ± 1.27† |
| | 12 | 0.9 ± 1.37 | 1.4 ± 1.31† | 1.7 ± 1.16† | 1.5 ± 1.24† |

Patients' assessment

| | | | | | |
|---|---|---|---|---|---|
| Reported improvement from | 4 | 59 (46%) | 62 (50%) | 63 (51%) | 125 (50%) |
| baseline (%) | 8 | 49 (53%) | 53 (72%) | 56 (68%) | 109 (70%) |
| | 12 | 49 (51%) | 54 (76%) | 51 (55%) | 105 (66%) |
| Mean change from baseline | 4 | 1.2 ± 1.27 | 1.3 ± 1.33 | 1.4 ± 1.20 | 1.4 ± 1.26 |
| | 8 | 1.3 ± 1.25 | 1.7 ± 1.26 | 1.8 ± 1.26 | 1.8 ± 1.25 |
| | 12 | 1.5 ± 1.29 | 1.9 ± 1.39 | 1.7 ± 1.09 | 1.8 ± 1.25 |

*Improvement was defined as patients who were moderately or markedly improved.
†p < 0.05 compared with usual care.

1. A method for the treatment of congestive heart failure in a subject in need thereof, said method comprising the serial and intermittent administering of a therapeutically effective dose of natriuretic peptide to said subject.

2. The method of claim 1 wherein said serial and intermittent administering comprises administering of a plurality infusions of said natriuretic peptide to said subject over a period of not less than 4 weeks, each of said infusions being spaced apart over a period of 1 to 14 days.

3. The method of claim 1 wherein said subject exhibits decompensated symptoms of congestive heart failure.

4. The method of claim 3 wherein said subject exhibits chronic symptoms of decompensated congestive heart failure.

5. The method of claim 1, wherein said natriuretic peptide is selected from the group consisting of an atrial natriuretic peptide (ANP), a B-type natriuretic peptide (BNP) and a C-type natriuretic peptide (CNP).

6. The method of claim 1, wherein the said natriuretic peptide is B-type natriuretic peptide.

7. The method of claim 6, wherein said B-type natriuretic peptide (BNP) is selected from the group consisting of human BNP, pig BNP, rat BNP, and chicken BNP.

8. The method of claim 6, wherein said natriuretic peptide is a human B-type natriuretic peptide.

9. The method of claim 6, wherein said natriuretic peptide is a recombinant B-type natriuretic peptide.

10. The method of claim 1, wherein said natriuretic peptide comprises a derivative of natriuretic peptide.

11. The method of claim 10 wherein said derivative comprises a derivative of atrial natriuretic peptide (ANP), B-type natriuretic peptide (BNP) or C-type natriuretic peptide (CNP).

12. The method of claim 10 wherein said derivative exhibits biological activity relative to one or more receptors of ANP, BNP, or CNP.

13. The method of claim 1, wherein said natriuretic peptide is atrial natriuretic peptide (ANP).

14. The method of claim 13, wherein said atrial natriuretic peptide (ANP) is selected from the group consisting of frog ANP and human ANP.

15. The method of claim 13, wherein said atrial natriuretic peptide (ANP) is human ANP.

16. The method of claim 13, wherein said atrial natriuretic peptide (ANP) is recombinant ANP.

17. The method of claim 1, wherein said natriuretic peptide is C-type natriuretic peptide (CNP).

18. The method of claim 17, wherein said C-type natriuretic peptide (CNP) is selected from the group consisting of chicken CNP, rat CNP and human CNP.

19. The method of claim 17, wherein said C-type natriuretic peptide (CNP is a human C-type natriuretic peptide.

20. The method of claim 17, wherein said natriuretic peptide is a recombinant C-type natriuretic peptide.

21. The method of claim 1, wherein each administration of said natriuretic peptide is by bolus followed by prolonged infusion.

22. The method of claim 1, wherein said natriuretic peptide is administered by bolus.

23. The method of claim 1, wherein said natriuretic peptide is administered by a combination of bolus and infusion.

24. The method of claim 21, wherein said natriuretic peptide is administered by bolus at a dose of not less than 2 μg/kg body weight.

25. The method of claim 21, wherein said natriuretic peptide is administered by serial infusion at a dose less than 0.01 μg/kg body weight.

26. The method of claim 1, wherein said natriuretic peptide is administered by intermittent infusion at a dose less than 0.01 μg/kg body weight.

27. The method of claim 1, wherein said natriuretic peptide is administered by intravenous, intramuscular, subcutaneous, intradermal, intrasternal, intraperitoneal or intra-articular injection.

28. The method of claim 1, wherein the said natriuretic peptide is administered prophylactically to said subject.

29. The method of claim 2, wherein each of said infusions of natriuretic peptide is administered to the subject over a period of 4-6 hours.

30. The method of claim 2, wherein said natriuretic peptide is administered to said mammal at an infusion rate of 0.00125 μg/kg body weight/min to 0.01 μg/kg body weight/min.

31. The method of claim 32, wherein said natriuretic peptide is administered to said subject at an infusion rate of 0.005 μg/kg body weight/min.

32. The method of claim 1, wherein said therapeutically effective dose comprises 0.25 μg/kg body weight to 1.75 μg/kg body weight of said natriuretic peptide.

33. A method to prevent or delay onset of acutely decompensated congestive heart failure requiring hospitalization in a subject suffering from chronic congestive heart failure, said method comprising serial and intermittent administration of a natriuretic peptide to the subject.

34. A method for preventing death or the need for hospitalization in a subject with congestive heart failure, said method comprising the serial and intermittent administrating of a therapeutically effective amount of natriuretic peptide to said subject.

35. A method for preventing the progression of congestive heart failure in a subject diagnosed therewith, said method comprising the serial and intermittent administering of a therapeutically effective amount of natriuretic peptide to said subject.

36. A method of treating congestive heart failure in a subject in need thereof, said method comprising the steps of diagnosing said subject as having an RAS score of ≧4 and administering a therapeutically effective amount of natriuretic peptide to said subject through a plurality of independent serial and intermittent infusions.

37. A method of treating congestive heart failure in a subject in need thereof, said method comprising the steps of diagnosing said subject as having renal insufficiency and administering a therapeutically effective amount of natriuretic peptide to said subject through a plurality of independent serial and intermittent infusions.

38. The method of claim 40 wherein said subject is diagnosed as having elevated serum creatinine levels greater than about 2.0 mg/dL

39. A method of treating congestive heart failure in a subject in need thereof, said method comprising the steps of diagnosing said subject as NYHA class III and exhibiting renal insufficiency; and administering a therapeutically effective amount of natriuretic peptide to said subject through a plurality of independent serial and intermittent infusions.

40. A method of treating congestive heart failure in a subject in need thereof, said method comprising the steps of diagnosing said subject as class IV NYHA and administering a therapeutically effective amount of natriuretic peptide to said subject through a plurality of independent serial and intermittent infusions.

41. A method of lowering aldosterone levels in a subject having congestive heart failure, said method comprising administering a therapeutically effective amount of natriuretic peptide to said subject through a plurality of independent serial and intermittent infusions.

42. A method of lowering endothelin 1 levels in a subject having congestive heart failure, said method comprising administering a therapeutically effective amount of natriuretic peptide to said subject through a plurality of independent serial and intermittent infusions.

43. A method to prevent cardiac remodeling in a subject diagnosed with congestive heart failure, said method comprising the administering of a therapeutically effective amount of natriuretic peptide to said subject through a plurality of independent serial and intermittent infusions.
